(19) United States
(12) Patent Application Publication
(10) Pub. No.: US 2003/0129219 A1
(43) Pub. Date: Jul. 10, 2003

(54) Self-emulsifying matrix type trandermal preparation

(76) Inventors: Chung Il Hong, Chicago, IL (US); Hee Jong Shin, Pucheon-city (KR); Min Hyo Ki, Cheonan-city, Choongchug-namdo (KR); Seok Kyu Lee, Choongchug-namdo (KR); Don Sun Kweon, Seoul (KR)

Correspondence Address: Richard L Byrne, 700 Koppers Building, 436 Seventh Avenue, Pittsburgh, PA, 15219-1818, US

(21) Appl. No.: 10/239,529

(22) PCT Filed: Mar. 29, 2001
(86) PCT No.: PCT/KR01/00509

(30) Foreign Application Priority Data
Mar. 29, 2000 (KR) 2000/16257

(51) Int. Cl. A61K 9/70

(52) U.S. Cl. 424/449

(57) ABSTRACT

The present invention relates to a novel pharmaceutical composition of a self-emulsifying matrix preparation, which is a preparation for transmucosal or transdermal absorption in which a self-emulsifying drug delivery system is grafted to a polymeric matrix preparation. For this, fatty alcohol, fatty acid or their derivatives of 6 to 20 carbon atoms having a drug absorption-accelerating action through the skin or mucous membrane is used as an oil phase. Also, to increase the drug content in the matrix, a liquid phase material having a boiling point of 100° C. or more is used as a solution adjuvant. Using such materials, the self-emulsifying system with a surfactant is prepared. A hydrophilic or hydrophobic polymer is added and dissolved in the self-emulsifying system, and the resulting mixture is dried to prepare the matrix preparation containing the self-emulsifying system. The self-emulsifying matrix preparation thus prepared maintains a constant drug-releasing rate during its application period by virtue of its excellent stability and exhibits an extraordinarily high skin-absorption rate.

### TECHNICAL FIELD

The present invention relates to a self-emulsifying matrix type transmucosal or transdermal preparation, which is capable of providing improved absorbability of drug substances through the mucous membrane or skin with minimized irritation.

### BACKGROUND ART

Up to now, a self-emulsifying drug delivery system has been conventionally applied to a preparation for oral or parenteral administration. A self-emulsifying system is characterized by being able to form spontaneously a thermodynamic-stable and homogeneous mixture when a small amount of surfactant is added to the binary system consisting of an aqueous phase and an oil phase. On the other hand, a classical emulsion, which appears to be opaque or milky, is thermodynamically unstable and cannot be formed spontaneously, so it requires great energy over the threshold for its formation. Further, the average particle size of a classical emulsion increases gradually as time goes by. However, a self-emulsifying system maintains a constant particle size regardless of time and appears to be transparent or translucent because it is thermodynamically stable.

There have been several attempts to apply a self-emulsifying system to a transmucosal or transdermal preparation. For example, Japan Patent Publication No. 03-127744 discloses a self-emulsifying system containing limonene as an oil phase for use in a transmucosal and transdermal preparation.

Also, U.S. Pat. No. 5,654,337 discloses a self-emulsifying system containing lecithin or phospholipid or their mixture as an oil phase. U.S. Pat. No. 5,759,566 discloses a self-emulsifying preparation in which poloxamer is added to form a gel at body temperature. U.S. Pat. No. 5,948,825 discloses a w/o microemulsion for use in a transmucosal or transdermal preparation, in which aqueous-phase droplets containing a physiologically active substance with low absorbability are dispersed in an oil-phase medium through the combination of plural surfactants.

The above-described patents have a common point with the present invention in that they design a self-emulsifying system including microemulsion to produce a transmucosal and transdermal preparation. However, the present invention differs from them to be applied in the form of liquid or semi-solid such as an ointment or a gel for the purpose of the sustained release of drug substances. None of them suggest a self-emulsifying matrix type preparation like the present invention that can contain an active substance in high concentration by combining a self-emulsifying system and a polymer matrix structure.

### DISCLOSURE OF THE INVENTION

The object of the present invention is to provide a self-emulsifying matrix type preparation, which is capable of containing a pharmacologically active substance in high concentration and avoiding the collapse of the self-emulsifying system caused by the volatilization of a solvent during a dry-forming process, for use in a highly permeable and non-irritable transdermal and transmucosal drug delivery system. After efforts to design such a self-emulsifying matrix preparation, the present inventors finally could present the solution of two technical problems involved in the prior arts and made the present invention on the basis of it.

First, for a matrix type preparation, it is necessary to mix a polymer solution and a self-emulsifying system, and then subject the resulting mixture to a dry-forming process. In this step, it is important that the precipitation of a drug substance or an oil component should not occur during those mixing and dry-forming process. In other words, no phase separation (white turbidity) including precipitation should not be observed in both the mixture containing a large amount of solvent and water before the drying process and the dried matrix containing a small or no amount of solvent and water after the drying process. The present inventors found through repeated experiments that the composition of a self-emulsifying system can overcome the above problem. Especially it is the most preferable solution to make a self-emulsifying system, which can form the self-emulsification at the aqueous phase ratio of 0 to 60%. When the composition was combined with a polymer matrix formulation, no phase separation took place in both the mixture before the drying process and the matrix after the drying process, and a homogeneous self-emulsifying matrix could be obtained.

Secondly, the inventors have prepared a self-emulsifying system capable of containing an active substance in high concentration. According to the present invention, the self-emulsifying system comprises a non-polar absorption promoter such as fatty acid, fatty alcohol, or derivatives thereof as an oil-phase, and a co-solvent with a boiling point of 100° C. or higher, which can increase the solubility of insoluble active substances, such as diethyleneglycol monoethyl ether, N-methyl 2-pyrrolidone, dimethylsulphoxide.

Therefore, the present invention provides the composition of a self-emulsifying matrix type preparation for a transdermal or transmucosal application as follows,

1) a polymer matrix,

2) an oil phase,

3) at least one co-solvent,

4) at least one surfactant,

5) an aqueous phase (water), and

6) at least one pharmacologically active substance.

The matrix preparation can be prepared by drying the resulting mixture solution after mixing and dissolving the components.

The matrix preparation according to the present invention may be provided with release films to be removed upon use, on both sides or on only the side applied to the body membrane. And a backing layer, through which the pharmacologically active substance cannot permeate, can be provided on the opposite side of the release film. The matrix preparation also may include additives such as antioxidants and preservatives, which are pharmaceutically acceptable.

As described previously, the present invention is characterized by the combination of the mixture of the components 2) to 4) and the aqueous phase (water) 5) at a certain ratio in order to form a self-emulsifying system. When water is added at the ratio of 0 to 60% to the total mixture, preferably 0 to 50%, the self-emulsification can be formed. The self-emulsification should be formed continuously over the range of at least 20%. For example, the mixture of components 2) to 4) is prepared and water is added to 40 g of the mixture little by little. If the self-emulsification is formed and maintained during the gradual addition of water in the amount of 10 g (20%) to 60 g (60%), it can be said that this mixture is a self-emulsifying system at the ratio of the aqueous phase of 20% to 60% and its continuous self-emulsifying range is 40%.

When the composition fails to accomplish the continuous self-emulsifying range of over 20% or the range is discontinuous, which indicates that a conventional emulsion, not a self-emulsifying system, is formed or the phase separation takes place, causing a precipitation. In case of that, it can cause instability due to the increase or decrease of water content by surroundings during the long-term storage. Further, upon the application to skin or mucous membranes, the drug may be precipitated by body fluids before the matrix releases the drug. These events commonly reduce the drug release rate considerably, resulting in lowering the absorption of the drug through skin or the mucous membrane.

In addition, in order to attain more constant skin penetration rate, it is desirable to select a polymer that can accord with the composition of the self-emulsifying mixture. According to the present invention, the polymer 1) to be used is variable depending on the content of an aqueous phase in the self-emulsifying mixture, as shown in Table 1. As the content of an aqueous phase is high, a hydrophilic polymer is used preferably. On the contrary, a hydrophobic polymer is used preferably as the content is low. When the self-emulsification is formed throughout the entire range of an aqueous phase, both hydrophilic and hydrophobic polymers can be used.

TABLE 1
Selection of polymers in accordance with the self-
emulsifying system

| Content of aqueous phase | |
|---|---|
| forming a self-emulsification | Selectable polymers |
| 0 to 40% | Hydrophobic polymers |
| 20 to 60% | Hydrophilic polymers |

The term “self-emulsifying” as used herein means that a transparent or translucent homogeneous product can be formed spontaneously without precipitation or phase separation when an oil phase, a surfactant, a co-solvent, and an aqueous phase are mixed together. The term “self-emulsifying system” as used herein refers to a homogeneous mixture being able to make the above-described phenomenon. The term “self-emulsification” as used herein refers to the obtainable resultant through the “self-emulsifying” phenomenon. The term “self-emulsifying matrix” as used herein refers to the polymer matrix preparation containing the self-emulsifying system according to the present invention.

The components of the preparation according to the present invention will be further explained in detail.

The polymer 1) is a component forming a solid body in the matrix type. The polymer to be used in the present invention includes a hydrophilic polymer able to be dissolved in water or a lower alcohol having up to 4 carbon atoms. The hydrophilic polymer may comprise at least one selected from the group consisting of hydroxyethyl cellulose, hydroxypropyl cellulose, hydroxypropylmethyl cellulose, hydroxypropylmethyl cellulose phthalate, cellulose acetate phthalate, carboxymethyl cellulose, polyethyleneoxide, chitosan, alginic acid, gelatin, polyvinylpyrrolidone, polyvinyl alcohol, poly (methyl vinyl ether/maleic anhydride), poly (vinylpyrrolidone/polyvinylacetate), polyacrylamide and pharmaceutically acceptable salts thereof. The derivatives of above-described polymer, which are prepared by binding hydroxyl group, amine group, alkyl group or composite thereof to the polymer, also can be used. Further, according to the present invention, a hydrophobic polymer, which has a low aqueous solubility and can be dissolved in a nonpolar solvent such as a lower alcohol having up to 4 carbon atoms, ethyl acetate, hexane, etc., can be used as a component 1). The hydrophobic polymer may comprise at least one selected from the group consisting of polymethacrylate, polyvinyl acetate, polyvinylacrylate, polyacrylate, silicone, polyisobutylene and pharmaceutically acceptable salts thereof. The polymethacrylate may include polyalkylmethacrylate, polymethylaminoethylmethacrylate and polymethacrylic acid ester. The polyacrylate may include polymers derived from monomers of alkylacrylic acid, nitrilacrylic acid and hydroxyalkylacrylic acid. The silicone may include polymers derived from a monomer of dimethylsiloxan and silicone resin. The polyisobutylene may include polymers derived from monomers of isobutylene and may include butyl rubber.

The oil phase 2) is a component having both a polar and a nonpolar moiety to act as an absorption promoter through skin and the mucous membrane. The polar moiety consists of carboxyl group, hydroxyl group, polyhydric alcohol and polyoxyethylene and the nonpolar moiety consists of alkyl chains. These two moieties are chemically bonded by means of ester or ether bond. Polyhydric alcohol may include propyleneglycol, glycerin, polyethyleneglycol resulted from polymerization of 1 to 5 ethyleneglycol. Specific examples of the oil phase may include at least one selected from a group consisting of saturated or unsaturated fatty acids having 6 to 20 carbon atoms, or its ester with one selected among polyhydric alcohols such as propyleneglycol, glycerin and polyethyleneglycol, or at least one selected from the group consisting of saturated or unsaturated fatty alcohol having 6 to 20 carbon atoms, or its ether with polyoxyethylene. Vegetable and animal oil containing the fatty acid, fatty alcohol, and derivatives thereof also can be included in the oil phase of the present invention.

The co-solvent 3) may include at least one selected from a group consisting of diethyleneglycol monoethyl ether, N-methyl 2-pyrrolidone, dimethylsulphoxide, propyleneglycol, etc. The co-solvent according to the present invention is distinguishable from conventional co-solvents such as ethyl alcohol, isopropyl alcohol, etc. in that it is in liquid state at room temperature and has a boiling point of over 100° C. (ex. Diethyleneglycol monoethyl ether: 202° C., N-methyl 2-pyrrolidone: 206° C., dimethylsulphoxide: 189° C., propyleneglycol: 188.2° C.) not to volatilize upon a dry-forming process of the matrix. Also, the co-solvent is miscible with the components 1) to 5) and can dissolve any material having a HLB (Hydrophilic-Lipophilic Balance) of 1 to 20. In particular, diethyleneglycol monoethyl ether, N-methyl 2-pyrrolidone, and dimethylsulphoxide can increase dramatically the solubility of insoluble materials, as compared to other co-solvents and surfactants (See Table 2). For this reason, they are useful in transmucosal and transdermal formulations requiring a high load of drug (pharmaceutically active substance). Experiments to evaluate the capability of several co-solvents to dissolve flurbiprofen and estradiol widely known as insoluble materials were carried out. The results are shown in Table 2. Using these co-solvents according to the present invention, it is possible to prepare a self-emulsifying matrix containing drug substances in high concentration.

TABLE 2
Capability of co-solvents and surfactants to dissolve
the insoluble materials

| | | Drug solubility (mg/ml) | |
|---|---|---|---|
| | | Flurbiprofene | Estradiol |
| Co-solvents | Diethyleneglycol | ≧500 | ≧200 |
| | monoethyl ether | | |
| | N-methyl | ≧2,000 | ≧1,000 |
| | 2-pyrrolidone | | |
| | Dimethylsulphoxide | ≧3,500 | ≧1,000 |
| | Propyleneglycol | ≧50 | ≧10 |
| | Ethanol | ≧250 | ≧20 |
| | Isopropyl alcohol | ≧167 | — |
| Surfactants | PEG*-6 glyceryl | ≧25 | — |
| | oleate (HLB 3/4) | | |
| | PEG-4 glyceryl | ≧50 | <10 |
| | caprylate (HLB 5) | | |
| | PEG-8 glyceryl | ≧50 | — |
| | oleate (HLB 6) | | |
| | POE*-2 oleic acid | ≧167 | <10 |
| | Ether (HLB 7.5) | | |
| | Polyglycolated | ≧100 | <10 |
| | glyceryl caprylate | | |
| | (HLB 10) | | |
| | PEG-8 glyceryl | ≧250 | ≧50 |
| | caprylate (HLB 14) | | |
| | Poloxamer | ≧500 | ≧10 |

*PEG: polyethyleneglycol, POE: polyoxyethylene

Examples of the surfactant 4) may include at least one selected from a group consisting of glycerin fatty acid ester, propyleneglycol fatty acid ester, polyethylene-glycol fatty acid ester, polyethyleneglycol glycerin fatty ester, polyoxyethylene fatty acid ether, fatty acid sorbate (Span), polyoxyethylene fatty acid sorbate (Tween), polyoxyethylene hydride castor oil (Cremophor), polyoxyethylene polyoxypropylene polymer (Poloxamer), etc. having a HLB of at least 3.

As an aqueous phase 5), water is used. In case that water is not added, the matrix preparation of the present invention will absorb moisture from the mucous membrane or skin to form a self-emulsification on the surface of the matrix.

The pharmacologically active substances 6) which can be applied to the self-emulsifying matrix according to the present invention include, but are not limited to, therapeutic agents for the circulating system such as nitroglycerin, isosorbide dinitrate, clonidine, prazosin, etc., therapeutic agents for the respiratory system such as clenbuterol, albuterol, salbutamol, etc., therapeutic agents for the mental disease such as methadon, fentanyl, codeine, etc., steroidal drug such as estradiol, progestin, testosterone, etc., analgesics and non-steroidal anti-inflammatory agents such as acetaminophen, ketoprofen, flurbiprofen, piroxicam, ketorolac, etc., anti-smoking agents such as nicotine, anti-cancer agents such as fluorouracil, etc., therapeutic agents for erectile dysfunction such as papaverine, alprostadil, yohimbin, etc., anti-histamine agents such as chlorpheniramine, etc., agents for the autonomic nervous system such as physostigmine, adrenolole, arecoline, etc., anti-bacterial or fungal agents such as amoxicillin, tetracycline, neomycin, fumagillin, therapeutic agents for cutaneous disorders such as retinoic acid, tocopherol, resorcinol, etc., anti-emetic agents such as ondansetron, meclizine, scopolamine and pharmaceutically acceptable salts thereof.

When the matrix type preparation is prepared according to the present invention, a backing layer and a release film may be further provided for storage and transfer. The backing layer should be made of materials through which the self-emulsifying system containing the drug dispersed in the matrix cannot permeate while protecting the matrix against surroundings and preventing the sudden increase or decrease of moisture content within the matrix to maintain a constant release rate. The materials may be polymers including polyolefin such as polyethylene or polypropylene, polyvinylchloride, polyethylenevinylacetate, polyethylenephthalate, polyurethane, etc. If necessary, films with a laminated metallic foil can be used. In addition, non-woven fabrics made of polyethylene, polypropylene, ray-on and artificial silk can be used. The non-woven fabrics can be a two-layer structure comprising an inactive film, which is coated to prevent the drug from diffusing inversely through them.

The release film is an inactive film attached to the surface of matrix, which is applied to the mucous membrane or skin and can be removed upon use. It is used for the assurance of stability during the storage period. For Example, a suitable release film may include a polyethylene or polyester film with or without silicone coating.

Now, the preparation procedure of a matrix type formulation using the above-described components according to the present invention will be described in detail.

An oil phase, a co-solvent and a surfactant are mixed together to form a transparent homogeneous solution. Upon adding the aqueous phase to the solution, a transparent or translucent self-emulsification in the form of liquid or gel is obtained. For example, oleic acid (FIG. 1) and propyleneglycol lauric acid ester (FIG. 2) as the oil phase, poloxamer 124 as the surfactant and diethyleneglycol diethyl ether as the co-solvent are mixed and water is added little by little to examine the self-emulsifying region. The results are shown in FIGS. 1 and 2. In FIGS. 1 and 2, “Oil” represents the oil phase and “Surfactant” represents the mixture (1:1) of a surfactant and a co-solvent. The reference lines 1), 2), 3), 4), and 5) represent a self-emulsifying system comprising the oil and the surfactant at the ratio of 7:3, 5:5, 3:7, 2:8, and 1:9, respectively. It is observed that a self-emulsifying system is formed within the water region of over 20% to 60% under the reference lines 2) to 5), which shows the features of the present invention. A polymer solution is separately prepared by dissolving a polymer into purified water or volatile solvent (a lower alcohol having up to 4 carbon atoms, or acetone, ethylacetate, hexane, etc.) to form a transparent solution. The polymer used is selected in accordance to Table 1, and a pharmaceutically acceptable cross-linking agent and plasticizer can be added depending on the selected polymer. The self-emulsifying system and the polymer solution are mixed, and then the pharmacologically active substance is added to the mixture. The resulting mixture solution is formed in a semisolid state with a thickness of about 20 to 600 μm using a Lab coater, and dried for several minutes to 1 hour at room temperature to 130° C. to prepare a self-emulsifying matrix with a thickness of 10 to 300 μm. Here, UV ray may be radiated within 1 hour depending on the polymer, if necessary. Through these processes, any volatile solvent such as a lower alcohol and acetone is totally removed. After drying obtained transparent self-emulsifying matrix contains a given amount of self-emulsifying system, water and pharmaceutically active substances dispersed homogeneously over the network structure of the polymer. When an aqueous phase is excluded, a self-emulsification can be formed by absorbing moisture within the body such as sweat or saliva upon the application to the mucous membrane or skin.

The matrices prepared according to the present invention have a transparent or translucent appearance and differ in their adhesive strength and cohesive strength depending on polymers used. Therefore, it is possible to prepare a self-emulsifying matrix suitable for various applications.

The self-emulsifying matrix finally applied according to the present invention is advantageous in that it contains self-emulsified particles within the network of the polymer and provides a high porosity, which is a favorable condition for diffusion through the matrix compared to solid particles prepared only from a co-solvent. In addition, the oil phase contained in the matrix of the present invention can act as an absorption promoter itself to improve the penetration rate of the drug through the mucous membrane or skin, and the aqueous phase can hydrate the corneum to increase the fluidity of stratum corneum, resulting in further increase of transdermal penetration of the drug.

Furthermore, since the self-emulsifying matrix is more stable than a conventional emulsion, it is possible to avoid the drug precipitation (crystallization) and maintain the constant drug release rate. In addition, since the absorption promoter, a main factor causing the mucous membrane and skin irritation, is substituted with the oil phase, it is possible to reduce the additional use of the absorption promoter and minimize the skin irritation.

### BRIEF DESCRIPTION OF THE DRAWINGS

The above objects, and other features as well as advantages of the present invention will become more apparent when taken in conjunction with the drawings, in which:

FIG. 1 is a tertiary diagram showing the self-emulsifying region of the self-emulsifying system containing oleic acid as an oil phase according to the present invention.

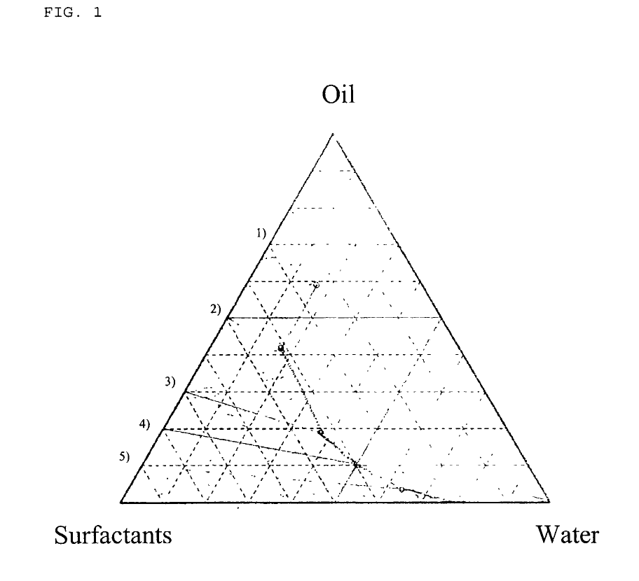

FIG. 2 is a tertiary diagram showing the self-emulsifying region of the self-emulsifying system containing propyleneglycol lauric acid ester as an oil phase according to the present invention.

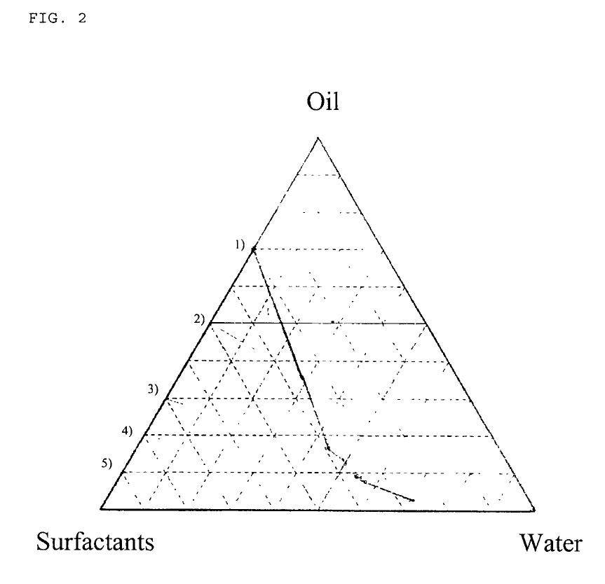

FIG. 3 is a graph showing the drug release pattern of the self-emulsifying matrix containing ketoprofen as an active agent according to the present invention.

FIG. 4 is a graph showing the drug release pattern of the self-emulsifying matrix containing diclofenac diethylammonium as an active agent according to the present invention.

FIG. 5 is a graph showing the permeation profile for the self-emulsifying matrix containing ketoprofen as an active agent according to the present invention.

FIG. 6 is a graph showing the permeation profile for the self-emulsifying matrix containing diclofenac diethylammonium as an active agent according to the present invention.

### BEST MODE FOR CARRYING OUT THE INVENTION

Now, preferred embodiments according to the present invention will be described in detail. However, the embodiments are for illustration not for restriction. The amount described in the specification and examples means % by weight unless described otherwise

### EXAMPLE 1

A self-emulsifying system with oleic acid as an oil phase is prepared to have a composition described below in Table 3.

TABLE 3

| Component | |
|---|---|
| Oleic acid | 20 |
| Diethyleneglycol | 40 |
| monoethyl ether | |
| Poloxamer 124 | 40 |
| Purified water | 0-110 |

Forty grams of Poloxamer as a surfactant and 40 g of diethyleneglycol monoethyl ether as a co-solvent were mixed. Then, 20 g of oleic acid as an oil-phase was added thereto while uniformly mixing to form a self-emulsifying system. A continuous self-emulsification can be obtained when purified water was added with varying the amount within a range of 0 to 110 g, (0 to 55% as a portion of aqueous phase to the self-emulsifying system).

### EXAMPLES 2-5

Self-emulsifying systems were prepared to have compositions described in Table 4. As an aqueous phase, purified water was used

TABLE 4

| Component | Ex.2 | Ex.3 | Ex.4 | Ex.5 |
|---|---|---|---|---|
| Oleyl alcohol | 20 | 10 | | |
| Linoleyl alcohol | | | 20 | 10 |
| Glycerin(1) oleic acid | | 10 | | |
| ester | | | | |
| Glycerin(1) linoleic acid | | | | 10 |
| ester | | | | |
| Diethyleneglycol | 40 | 40 | 40 | 40 |
| monoethyl ether | | | | |
| Polyethyleneglycol(8) | 40 | | 40 | |
| glycerin(1) capric acid | | | | |
| ester | | | | |
| Cremophor RH40 | | 40 | | 40 |

One or two selected from oleyl alcohol, linoleyl alcohol, glycerin(1) oleic acid ester and glycerin(1) linoleic acid ester as described in the above table were mixed together to form an oil-phase. Further, diethyleneglycol monoethyl ether as a co-solvent, and polyethyleneglycol(8) glycerin(1) capric acid ester and cremophor RH40 as surfactants were mixed together as described in the above table and added to the oil-phase while mixing uniformly. Upon the addition of purified water, a self-emulsification was obtained in any case.

### EXAMPLES 6-9

Self-emulsifying systems were prepared to have compositions described in Table 5. As an aqueous phase, purified water was used

TABLE 5

| Component | Ex.6 | Ex.7 | Ex.8 | Ex.9 |
|---|---|---|---|---|
| Oleic acid | 20 | 10 | | |
| Linoleic acid | | | 20 | 10 |
| Glycerin(1) oleic acid | | 10 | | |
| ester | | | | |
| Glycerin(1) linoleic acid | | | | 10 |
| ester | | | | |
| Diethyleneglycol | 40 | 40 | 40 | 40 |
| monoethyl ether | | | | |
| Polyethyleneglycol(8) | 40 | | 40 | |
| glycerin(1) capric acid | | | | |
| ester | | | | |
| Poloxamer 124 | | 40 | | 40 |

One or two selected from oleic acid, linoleic acid, glycerin(1) oleic acid ester and glycerin(1) 15 linoleic acid ester as described in the above table were mixed together to form an oil-phase. Further, diethyleneglycol monoethyl ether as a co-solvent, and polyethylene glycol(8) glycerin(1) capric acid ester and poloxamer 124 as surfactants were mixed together as described in the above table and added to the oil-phase while mixing uniformly. Upon the addition of purified water, a self-emulsification was obtained in any case.

### EXAMPLES 10-13

Self-emulsifying systems were prepared to have compositions described in Table 6. As an aqueous phase, purified water was used

TABLE 6

| Component | Ex.10 | Ex.11 | Ex.12 | Ex.13 |
|---|---|---|---|---|
| Propyleneglycol(1) | 33 | | 33 | |
| lauric acid ester | | | | |
| Polyethyleneglycol(2) | | 20 | | 20 |
| oleic acid ester | | | | |
| N-methyl 2-pyrrolidone | 33 | 40 | 33 | 40 |
| Polyethyleneglycol(8) | | 40 | | 40 |
| glycerin(1) capric acid | | | | |
| ester | | | | |
| Poloxamer 124 | 34 | | 34 | |

Any one selected from propyleneglycol(1) lauric acid ester and polyethyleneglycol(2) oleic acid ester as described in the above table was used as an oil-phase. Further, N-methyl 2-pyrrolidone as a co-solvent, and polyethyleneglycol(8) glycerin(1) capric acid ester and poloxamer 124 as surfactants were mixed together as described in the above table and added to the oil-phase while uniformly mixing. Upon the addition of purified water, a self-emulsification was obtained in any case.

### EXAMPLES 14-17

Self-emulsifying systems were prepared to have compositions described in Table 7. As an aqueous phase, purified water was used.

TABLE 7

| Component | Ex.14 | Ex.15 | Ex.16 | Ex.17 |
|---|---|---|---|---|
| Squalene | 10 | 10 | | |
| Glycerin fatty acid(3) | | | 10 | 10 |
| ester | | | | |
| (MCT Oil) | | | | |
| Polyethyleneglycol(2) | 25 | | 25 | |
| oleic acid ester | | | | |
| Polyoxyethylene(2) oleic | | 20 | | 20 |
| acid ester | | | | |
| N-methyl 2-pyrrolidone | 25 | 35 | 25 | 35 |
| Polyglycerin(6) dioleic | 20 | | 20 | |
| acid ester | | | | |
| Tween 20 | 20 | 35 | 20 | 35 |

One or two selected from squalene, glycerin fatty acid(3) ester (MCT Oil), polyethyleneglycol(2) oleic acid ester and polyoxyethylene(2) oleic acid ester as described in the above table were mixed together to form an oil-phase. Further, N-methyl 2-pyrrolidone as a co-solvent, and polyglycerin(6) dioleic acid ester and Tween 20 as surfactants were mixed together as described in the above table and added to the oil-phase while uniformly mixing. Upon the addition of purified water, a self-emulsification was obtained in any case.

### EXAMPLE 18

Using the self-emulsifying system from Example 3, a self-emulsifying matrix was prepared. To 10 g of the self-emulsifying system from Example 3 was added 5 g of arecoline HBr as a drug. Sixty grams of polyethyleneoxide was dissolved into 30 g of water and 30 g of ethanol to form a polymer solution. This pre-polymer solution was added to the self-emulsifying system containing the drug to give a transparent viscous solution, which was then dried at 80° C. for 10 minutes to form a self-emulsifying matrix with a thickness of 50 μm. During the process of drying, UV ray may be irradiated for 5 minutes, if necessary.

### EXAMPLE 19

Using the self-emulsifying system from Example 4, a self-emulsifying matrix was prepared. To 10 g of the self-emulsifying system from Example 4 was added 1 g of estradiol as a drug. Fifteen grams of hydroxyethyl cellulose and 45 g of poly(methylvinylether/maleic anhydride) polymer were dissolved into 15 g of water and 25 g of ethanol to form a polymer solution. This pre-polymer solution was added to the self-emulsifying system containing the drug to give a transparent viscous solution. Ten grams of glycerin was added as a cross linking agent. Then, the final solution was dried at 80° C. for 10 minutes to form a self-emulsifying matrix with a thickness of 50 μm.

### EXAMPLE 20

Using the self-emulsifying system from Example 7, a self-emulsifying matrix was prepared. To 10 g of the self-emulsifying system from Example 7 was added 2 g of methadone as a drug. Fifty grams of polymethacrylate was dissolved into 10 g of water, 30 g of ethanol and 20 g of acetone to form a polymer solution. This pre-polymer solution was added to the self-emulsifying system containing the drug to give a transparent viscous solution. Ten grams of triethyl citric acid and 5 g of succinic acid were added as a plasticizing and a cross-linking agent, respectively. Then, the final solution was dried at 80° C. for 10 minutes to form a self-emulsifying matrix with a thickness of 50 μm.

### EXAMPLE 21

Using the self-emulsifying system from Example 9, a self-emulsifying matrix was prepared. To 15 g of the self-emulsifying system from Example 9 was added 3 g of ondansetron as a drug. Fifty grams of polyacrylate was dissolved into 45 g of ethanol and 15 g of ethylacetic acid to form a polymer solution. This pre-polymer solution was added to the self-emulsifying system containing the drug to give a transparent viscous solution, which was then dried at 80° C. for 10 minutes to form a self-emulsifying system matrix with a thickness of 50 μm.

### EXAMPLE 22

Using the self-emulsifying system from Example 1, a self-emulsifying matrix was prepared. To 15 g of the self-emulsifying system from Example 1 was added 10 g of ketoprofen as a drug. Fifty grams of polyacrylate was dissolved into 35 g of ethanol and 25 g of ethylacetic acid to form a polymer solution. This pre-polymer solution was added to the self-emulsifying system containing the drug to give a transparent viscous solution, which was then dried at 100° C. for 2 minutes to form a self-emulsifying matrix with a thickness of 80 μm.

### EXAMPLE 23

A self-emulsifying matrix was prepared following the same procedures as Example 22, except ketoprofen used in the amount of 15 g.

### EXAMPLE 24

A self-emulsifying matrix was prepared following the same procedures as Example 22, except ketoprofen used in the amount of 20 g.

### EXAMPLE 25

Using the self-emulsifying system from Example 10, a self-emulsifying matrix was prepared. To 15 g of the self-emulsifying system from Example 10 was added 20 g of diclofenac diethylammonium as a drug. Fifty grams of polyacrylamide was dissolved into 10 g of purified water and 50 g of ethanol to form a polymer solution. This pre-polymer solution was added to the self-emulsifying system containing the drug to give a transparent viscous solution, which was then dried at 100° C. for 2 minutes to form a self-emulsifying matrix with a thickness of 70 μm.

### EXAMPLE 26

A self-emulsifying matrix was prepared following the same procedures as Example 25, except diclofenac used in the amount of 30 g.

### EXAMPLE 27

A self-emulsifying matrix was prepared following the same procedures as Example 25, except diclofenac used in the amount of 40 g.

### COMPARATIVE EXAMPLE 1

A comparative mixture was prepared similarly to Example 3, except that diethyleneglycol monoethyl ether was replaced with the same amount of ethanol. When purified water was added, the mixture showed emulsion or suspension form. Using the comparative mixture, a comparative matrix was prepared following the same procedures as in Example 18.

### COMPARATIVE EXAMPLE 2

A comparative mixture was prepared similarly to Example 4, except that diethyleneglycol monoethyl ether was replaced with the same amount of ethanol. When purified water was added, the mixture showed a phase separation in a solution state. Using the comparative mixture, a comparative matrix was prepared following the same procedures as in Example 19.

### COMPARATIVE EXAMPLE 3

A comparative mixture was prepared similarly to Example 7, except that diethyleneglycol monoethyl ether was replaced with the same amount of ethanol. When purified water was added, the mixture showed emulsion or suspension form. Using the comparative mixture, a comparative matrix was prepared following the same procedures as in Example 20.

### COMPARATIVE EXAMPLE 4

A comparative mixture was prepared similarly to Example 9, except that diethyleneglycol monoethyl ether was replaced with the same amount of ethanol. When purified water was added, the mixture showed emulsion or suspension form. Using the comparative mixture, a comparative matrix was prepared following the same procedures as in Example 21.

### TEST EXAMPLE 1

The matrices prepared in Examples 18-21 and Comparative examples 1-4 were sealed up in vinyl packages to avoid contacting with moisture. The matrices in packages were kept in a chamber with controlled temperature and humidity (40° C., 75% RH) for a predetermined period of time. After the storage for 1 and 2 months, the matrices were checked for the changes of appearance and the crystallization of the active ingredients. The results are shown in Table 8.

TABLE 8

| | Initial | | 1 month | | 2 months | |
|---|---|---|---|---|---|---|
| | Appear- | Crystal- | Appear- | Crystal- | Appear- | Crystal- |
| | ance | lization | ance | lization | ance | lization |
| Ex. 18 | Trans- | X | Trans- | X | Trans- | X |
| | parent | | parent | | parent | |
| Com. Ex. 1 | Turbid | X | Turbid | X | Turbid | X |
| Ex. 19 | Trans- | X | Trans- | X | Trans- | X |
| | parent | | parent | | parent | |
| Com. Ex. 2 | Turbid | O | Turbid | O | Turbid | O |
| Ex. 20 | Trans- | X | Trans- | X | Trans- | X |
| | parent | | parent | | parent | |
| Com. Ex. 3 | Turbid | X | Turbid | O | Turbid | O |
| Ex. 21 | Trans- | X | Trans- | X | Trans- | X |
| | parent | | parent | | parent | |
| Com. Ex. 4 | Trans- | X | Turbid | O | Turbid | O |
| | parent | | | | | |

As seen from the above results, all the matrices prepared in Examples 18-21 according to the present invention showed transparent appearance. Even after 2 months, crystallization was not observed. Meanwhile, the matrices prepared in Comparative Examples 1-4 showed turbid appearance at the initial state or after 1 month. Further, crystals were observed in the matrices of Comparative Examples 2-4 with magnifying power of 100. It indicates that they are physically unstable not to maintain the initial state of solution or emulsion and drug precipitation occurred.

### TEST EXAMPLE 2

For the self-emulsifying matrices prepared in Examples 22-27 according to the present invention, the amount of drug diffused from the matrices toward a medium was measured by carrying out the drug diffusion (release) test using Microette Topical & Transdermal Diffusion Cell System. In particular, the matrices were cut into a circle (diameter: 15 mm), attached to a cellulose acetate film, fixed with dosage wafer and mounted on a Franz type vertical cell (diameter: 15 mm, effective volume: 7 ml). Physiological saline (pH 7.4) was used as a medium. Samples were taken automatically at predetermined intervals (5, 10, 20 and 30 minutes, and 1, 2, 4, 6, 9 and 12 hour(s)) and analyzed by the known HPLC method.

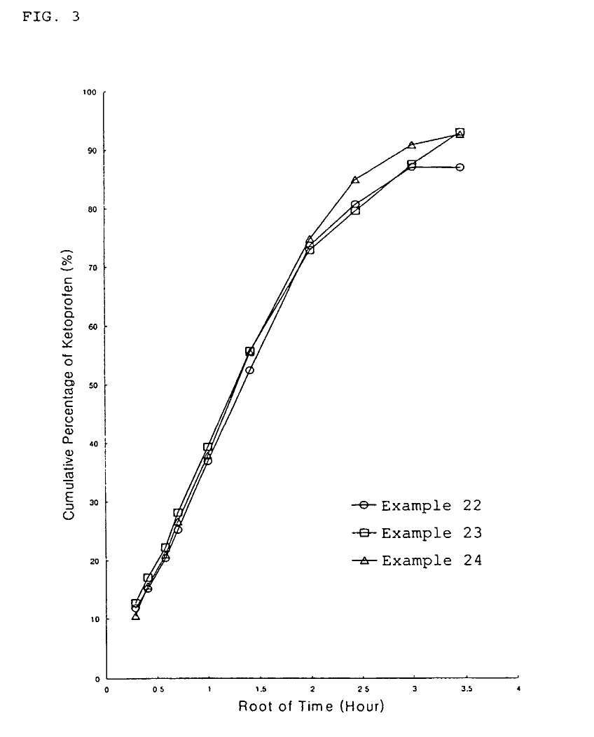

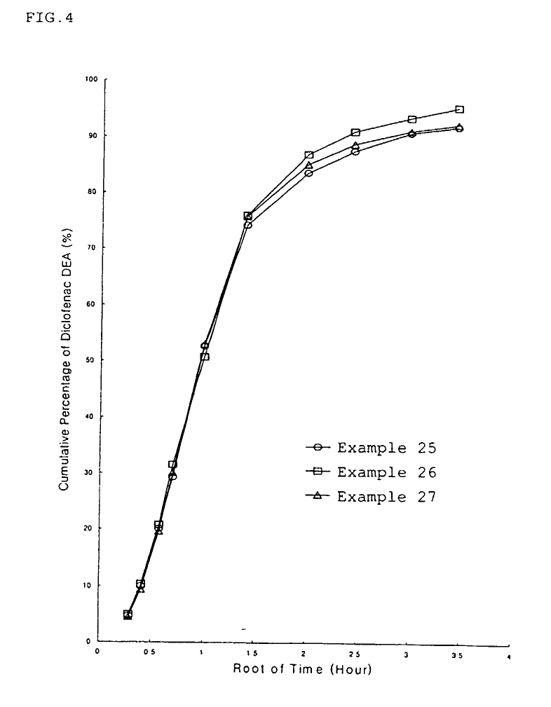

As a result, diffusion patterns for the matrix samples from Examples 22-24 were shown in FIG. 3. And diffusion patterns for the matrix samples from 25-27 were shown in FIG. 4. As known to researchers in this field, it is reported that the diffusion of active ingredients (drugs) from a matrix complies to Higuchi's Equation (T^{1/2} Order) as follows:

dQ/dT=[ACₛD/2T]^{1/2} Eq. 1)

Q=K˜Root T Eq. 2)

Where Q is the amount of drug released, A is the initial amount of drug in unit volume of matrix, Cₛ is the solubility of drug in the matrix, D is the diffusion coefficient, and T is the time. When Eq. 1) is integrated and other constants except Q and T are assigned as K, Eq. 2) can be obtained. It indicates that the amount of drug released is proportional to the square root of time. It is generally known that there exists a linear relationship between the drug released or diffused from a single-layer matrix and the square root of time until 30% of the initial amount of drug is released, but the release rate decreases after then. However, from the self-emulsifying matrix according to the present invention, as shown in FIGS. 3 and 4, the release rate keeps up linearly until about 80% of the initial amount of drug is released. In other words, until most of the drug loaded in the matrix is released, a constant release rate can be attained. It is the unique feature of the self-emulsifying matrix according to the present invention without more addition of polymers or a release-controlling membrane.

### TEST EXAMPLE 3

For the self-emulsifying system matrices prepared in Examples 22-27 according to the present invention, the amount of drug permeated through skin toward a medium was measured by carrying out drug diffusion (release) test using Microette Topical & Transdermal Diffusion Cell System.

In particular, the matrices were cut into a circle (diameter: 15 mm), and attached to the corneum of skin removed from a hairless rat of 8 weeks old. The skin with the matrix is mounted on a Franz type vertical cell (diameter: 15 mm, effective volume: 7 ml) with a dosage wafer so that the side of dermis comes in contact with the medium. The medium is physiological saline solution (pH 7.4) and kept at 37° C. Samples were taken automatically at predetermined intervals (1, 3, 6, 9, 12, 18 and 24 hour(s)) and analyzed by the known HPLC method.

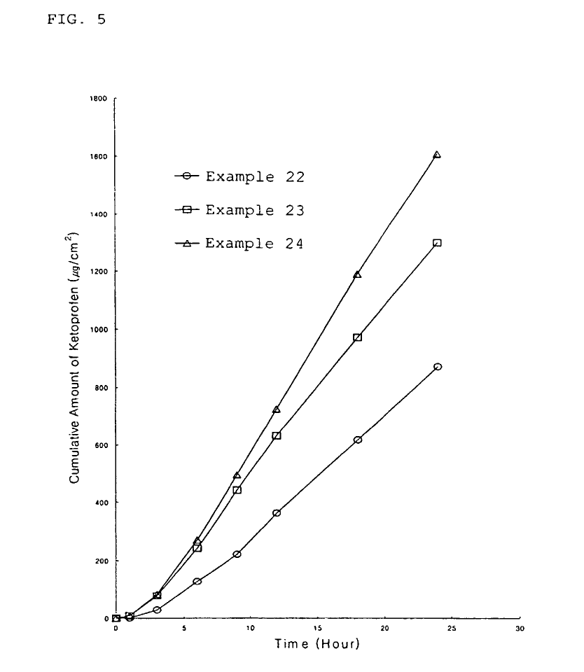

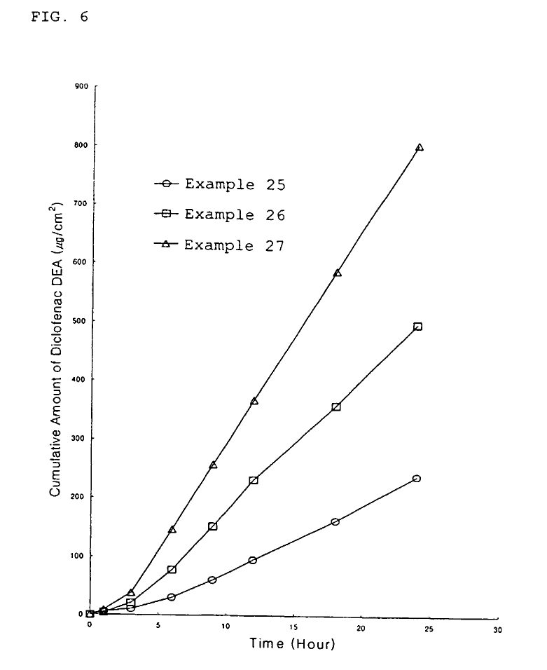

As a result, the permeation profiles for the matrix samples from Examples 22-24 and Examples 25-27 are shown in FIG. 5 and FIG. 6, respectively.

The amount of drug permeated through skin indicates the amount of drug absorbed through skin, which is closely associated with the activity of drug. Meanwhile, after a lag time the curve shows a constant slope in both FIGS. 5 and 6. The slope means the percutaneous absorption rate (Flux: percutaneous absorption amount per a unit area per a unit time, μg/cm²/hr). As the flux is higher, the higher activity of drug can be expected, and as the lag time is shorter, more rapid activity can be expected. From the results shown in FIGS. 5 and 6, the self-emulsifying system matrices containing ketoprofen prepared in Examples 22 to 24 show an excellent percutaneous absorption rate of 40.7 μg/cm²/hr up to 73.7 μg/cm²/hr and a favorable lag time with 1 to 2 hours, and Examples 25 to 27 show an excellent percutaneous absorption rate of 11.0 μg/cm²/hr to 36.5 μg/cm²/hr and a favorable lag time less than 2 hours.

Therefore, it is concluded that the self-emulsifying matrix according to the present invention accomplishes a constant drug release rate and a high transdermal permeation rate with good stability, so it satisfies the optimal requirements for transmucosal and transdermal absorption formulation.

Preparation of Various Types of Patches Containing the Self-Emulsifying Matrix

1. A Patch with Identical Front and Back (Releasing Film)

The final polymer solution from Example 18 was applied on a polyester film, which had been silicone-coated, with a knife using a Lab Coater. And then it was dried at 80° C. for 10 minutes to form a self-emulsifying matrix with a thickness of 50 μm. The other releasing film of identical material, a polyester film, which had been silicone-coated, was applied to the opposite side of the surface of the matrix where a polyester film was provided. The releasing film was applied using a roller to remove air. The resulting product was sealed and stored.

2. A Patch Supported on Polyurethane Film

The final polymer solution from one of Examples 22-27 was applied on a polyester film as a releasing film, which had been silicone-coated, with a knife using a Lab Coater. And then it was dried at 100° C. for 2 minutes to form a self-emulsifying matrix with a thickness of 80 μm (Examples 22-24) and 70 μm (Examples 25-27). Then, polyurethane film as a backing layer was applied to the opposite side of the surface of the matrix where the polyester film was provided. The backing layer was applied using a roller to remove air. The resulting product was sealed and stored.

3. A Patch Supported on Non-Woven Fabrics

The final polymer solution from one of Examples 22-27 was applied on a polyester film as a releasing film, which had been silicone-coated, with a knife using a Lab Coater. And then it was dried at 100° C. for 2 minutes to form a self-emulsifying matrix with a thickness of 80 μm (Examples 22-24) and 70 μm (Examples 25-27). Separately, a non-woven fabric as a backing layer was coated with an inactive film. For the inactive film, the same amount of polyisobutylene and hydrocarbon resin were mixed and dissolved into hexane to be 40% of solid. The solution was dried to form the inactive film with a thickness of 40 μm. Then, the film surface was subjected to a non-woven fabric and coated to prevent the drug from diffusing reversely into a non-woven fabric. The coated non-woven fabric was applied to the opposite side of the surface of the matrix where the polyester film was provided. The backing layer was applied using a roller to remove air. The resulting product was sealed and stored.

### INDUSTRIAL APPLICABILITY

As apparent from the above description, the present invention can provide a novel drug delivery system in which the self-emulsifying system is combined with a polymeric matrix for the transdermal or transmucosal administration of drug substances. The drug delivery system according to the present invention can assure the stability against moisture and a constant drug release rate. Therefore, it is possible to easily control the amount of drug absorbed through mucous membrane or skin and to prohibit the crystallization of the drug in the preparation. Further, through the self-emulsifying system containing an oil-phase and an aqueous phase without the phase separation, it is possible to develop pharmaceutical formulations by using the various polymers of the physicochemical properties suitable for each application.

1. A self-emulsifying matrix type transdermal and transmucosal preparation comprising:
1) a polymer matrix,
2) an oil phase
3) at least one co-solvent selected from a group consisting of diethyleneglycol monoethyl ether, N-methyl 2-pyrrolidone,
4) at least one surfactant, and
5) at least one pharmacologically active substance, in which the self-emulsifying system is dispersed over the polymer matrix.

2. The preparation according to claim 1, wherein the self-emulsifying system form the self-emulsification at the aqueous phase ratio of 0 to 60% and its continuous self-emulsifying range is at least 20%.

3. The preparation according to claim 2, wherein the polymer comprises at least one selected from the group consisting of hydroxyethyl cellulose, hydroxypropyl cellulose, hydroxypropylmethyl cellulose, hydroxypropylmethyl cellulose phthalate, cellulose acetate phthalate, carboxymethyl cellulose, polyethylene oxide, chitosan, alginic acid, gelatin, polyvinylpyrrolidone, polyvinyl alcohol, poly (methylvinyl ether/maleic anhydride), poly (vinylpyrrolidone/polyvinyl acetate), polyacrylamide, polymethacrylate, polyvinyl acetate, polyvinylacrylate, polyacrylate, silicone, polyisobutylene and pharmaceutically acceptable salts thereof.

4. The preparation according to claim 2, wherein the oil phase comprises at least one selected from the group consisting of saturated or unsaturated fatty acid having 6 to 20 carbon atoms, or its ester with one selected among polyhydric alcohols such as propyleneglycol, glycerin and polyethyleneglycol, or at least one selected from the group consisting of saturated or unsaturated fatty alcohol having 6 to 20 carbon atoms, or its ether with polyoxyethylene.

5. The preparation according to claim 2, wherein the pharmacologically active substance comprises at least one selected from therapeutic agents for the circulating system such as nitroglycerin, isosorbide dinitrate, clonidine, prazosin, etc., therapeutic agents for the respiratory system such as clenbuterol, albuterol, salbutamol, etc., therapeutic agents for the mental disease such as methadon, fentanyl, codeine, etc., steroidal drug such as estradiol, progestin, testosterone, etc., analgesics and non-steroidal anti-inflammatory agents such as acetaminophen, ketoprofen, flurbiprofen, piroxicam, ketorolac, etc., anti-smoking agents such as nicotine, anti-cancer medicines such as fluorouracil, etc., agents for erectile dysfunction such as papaverine, alprostadil, yohimbin, etc., anti-histamine agents such as chlorpheniramine, etc., agents for the autonomic nervous system such as physostigmine, adrenolole, arecoline, etc., anti-bacterial or fungal agents such as amoxicillin, tetracycline, neomycin, fumagillin, agents for cutaneous disorders such as retinoic acid, tocopherol, resorcinol, etc., anti-emetic agents such as ondansetron, meclizine, scopolamine and pharmaceutically acceptable salts thereof.

6. The preparation according to claim 3, wherein the polymethacrylate comprises at least one selected from the group consisting of polyalkylmethacrylate, polymethylaminoethylmethacrylate and ester of polymethacrylic acid, the polyacrylate comprises at least one selected from the group consisting of polymers derived from monomers of alkylacrylic acid, nitrilacrylic acid and hydroxyalkylacrylic acid, the silicone comprises at least one selected from the group consisting of polymer derived from a monomer of dimethylsiloxan and silicone resin, and the polyisobutylene comprises at least one selected from the group consisting of polymers derived from a monomer isobutylene and butyl rubber.

7. The preparation according to any one of claims 1 to 6, wherein the matrix formulation is provided with a release film to be removed upon use on the side applied to the body membrane, and another release film or a backing layer to protect the matrix on the opposite side of the release film bound.

8. The preparation according to any one of claims 1 to 6, wherein the formulation comprises further at least one additive selected from antioxidant, preservatives, etc., which are pharmaceutically available.
